(19) United States
(12) Patent Application Publication
(10) Pub. No.: US 2021/0321921 A1
(43) Pub. Date: Oct. 21, 2021

(54) FURAN SYRINGES AND BLOOD COLLECTION ITEMS

(71) Applicant: Shantu Patel, Carlsbad, CA (US)

(72) Inventor: Shantu Patel, Carlsbad, CA (US)

(21) Appl. No.: 16/851,064

(22) Filed: Apr. 16, 2020

(51) Int. Cl. A61B 5/15; C08L 67/02; A61B 5/153

(52) U.S. Cl. CPC A61B 5/150343; C08L 67/02; A61B 5/150351; A61B 5/15003; A61B 5/153; A61B 5/150244; A61B 5/150572

(57) ABSTRACT

A blood collection item comprises a furan resin selected from the group consisting of: poly (ethylene 2,5-furan dicarboxylate) (PEF), poly (butylene 2,5-furan dicarboxylate) (PBF), poly (trim ethylene furan dicarboxylate) (PTF), poly (propylene 2,5-furandicarboxylate) (PPF), and poly (neopentyl 2,5-furandicarboxylate) (PNF). The item may be a syringe having an injection-molded plunger and barrel, a blood collection tube, or a tube holder.

### BACKGROUND OF THE INVENTION

The present invention generally relates to medical devices made from furan resin and more specifically to furan syringes and blood collection items.

Environment and sustainability have become increasingly important factors in the design and specification of single use medical items across the world. In hospitals, pharmaceuticals, life sciences, and healthcare industries, safe disposal of articles after use is an important issue. Special considerations are given to selecting materials in the final design for disposable articles, so as to reduce the quantity of syringes items that enter waste streams. These facilities and industries must initiate environmentally safe disposal methods because they generate a large amount of the bio waste. Due to higher social responsibility and environmental concerns, corporations are being driven to produce more sustainable and environmentally safe products through government regulations, by institutional investors, and through consumer demand.

A vacutainer blood collection tube is a sterile glass or plastic tube with a colored rubber stopper creating a vacuum seal inside of the tube, facilitating the drawing of a predetermined volume of liquid. Blood collection or vacutainer tubes may contain additives designed to stabilize and preserve the specimen prior to analytical testing. Tubes may be available with a safety-engineered stopper, with a variety of labeling options and draw volumes. The color of the top may indicate the additives in the vial. The vacutainer has two main parts: the cylindrical tube and a rubber stopper. Tubes may include additives that mix with the blood when collected, and the color of each tube's rubber stopper or plastic cap may indicate which additives it contains. Blood collection tubes expire over time. The vacuum may be lost, and blood will not be drawn into the tube if the needle punctures the cap or stopper.

A tube holder is a device that assists the phlebotomist in blood collection, using a needle and blood specimen tubes. Blood collection can frequently lead to spurious hemolysis or erythrocyte injury. The tube holder should prevent erythrocyte injury in samples collected from a patient during a venipuncture procedure or a blood draw. It has a hollow cylindrical tube with a circular flange at the open end, distally. The proximal end is tapered with a tip to receive a needle that can be snapped on, or screwed on by a twisting motion. The tube holder design allows the phlebotomist to interchange the blood specimen tubes at easy, when multiple tubes have to be collected.

It would be desirable to have a syringe that includes a combination of bioplastic and biodegradable resins.

### SUMMARY OF THE INVENTION

In one aspect of the present invention, a blood collection item comprises a furan resin selected from the group consisting of: poly (ethylene 2,5-furan dicarboxylate) (PEF), poly (butylene 2,5-furan dicarboxylate) (PBF), poly (trim ethylene furan dicarboxylate) (PTF), poly (propylene 2,5-furandicarboxylate) (PPF), and poly (neopentyl 2,5-furandicarboxylate) (PNF).

In another aspect of the present invention, a syringe comprises a furan-based resin that includes at least one of: PEF, PBF, PTF, PPF, or PNF; an injection-molded barrel that includes the furan-based resin; and an injection-molded plunger that includes the furan-based resin.

### BRIEF DESCRIPTION OF THE DRAWINGS

FIG. 1 depicts an embodiment of a syringe with attached needle according to the present invention.

FIG. 2 depicts an embodiment of a needle and safety shield assembly according to the present invention.

FIG. 3 depicts an embodiment of a needle and safety shield in extended position according to the present invention.

FIG. 4 depicts an embodiment of a needle safety shield deployed with needle in the protective casing according to the present invention.

FIG. 5 depicts an embodiment of the blood collecting items, namely a collection tube with stopper, tube holder with a needle.

FIG. 6 depicts an embodiment of a tube holder.

FIG. 7 depicts an embodiment of a blood collection tube with a stopper.

FIG. 8 depicts an embodiment of blood collection tube with a cap.

### DETAILED DESCRIPTION

The preferred embodiment and other embodiments, which can be used in industry and include the best mode now known of carrying out the invention, are hereby described in detail with reference to the drawings. Further embodiments, features and advantages will become apparent from the ensuing description, or may be learned without undue experimentation. The figures are not necessarily drawn to scale, except where otherwise indicated. The following description of embodiments, even if phrased in terms of “the invention” or what the embodiment “is,” is not to be taken in a limiting sense, but describes the manner and process of making and using the invention. The coverage of this patent will be described in the claims. The order in which steps are listed in the claims does not necessarily indicate that the steps must be performed in that order.

The present invention generally relates to syringe, tube holder and blood collection tubes or vacutainers made from a plant based resin also known as furan-based polyesters.

Embodiments of the present invention may include a plant based furanpolyester, namely, poly (ethylene 2,5-furan dicarboxylate) (PEF), poly (butylene 2,5-furan dicarboxylate) (PBF), poly (trim ethylene furan dicarboxylate) (PTF), poly (propylene 2,5-furandicarboxylate) (PPF), and poly (neopentyl 2,5-furandicarboxylate) (PNF), to be used for sustainable single use medical items, such as syringes, tube holder and blood collection tubes also referred to as vacutainers.

In an embodiment, Furan moieties may have thermal stability characterized by a higher Tg and Tm. Furan moieties may include PNF, which has a low RAF fraction compared to poly (propylene 2,5- furandicarboxylate) [also called -furanoate] (PPF), ascribable to the two methyl side groups present in PNF glycol-sub-unit. PNF's mechanical characteristics, i.e., very high elastic modulus and brittle fracture, low resistance to environmental stress cracking, drop impact performance were found to be superior to those of PPF and PEF. Barrier properties to different gases, temperatures and relative humidity were evaluated. From the results obtained, PNF was showed to be a material with superior barrier performances, significantly superior with respect to PEF's ones. The furan moieties are not necessarily biodegradable, but may be recyclable. Because of these properties, and also because furan resins are plant based, compared to glass or plastic the furan syringes are considered far more sustainable. A syringe made from furan-based resin and polypropylene or glass could be recycled. The melting temperature for recycled plastic or polypropylene is 350 degrees F. and glass may be melted at 2900 degrees F. to recycle. Recycled furan requires 300 degrees to return back to resin, thus a more sustainable option compare to glass or marginally better than plastic, the latter are petroleum based and not plant based.

Embodiments of the present invention may provide a disposable syringe with a tubular needle cap and a flexible foldable needle safety shield, needle hub, tube holder and blood collection tubes made from plant derived sustainable furan resins, namely: poly (ethylene 2,5-furan dicarboxylate) (PEF), poly (butylene 2,5-furan dicarboxylate) (PBF), poly (trim ethylene furan dicarboxylate) (PTF), poly (propylene 2,5-furandicarboxylate) (PPF), and poly (neopentyl 2,5-furandicarboxylate) (PNF).

Embodiments may include a furan resin based syringe with attached needle and foldable and extendable protective safety shield assembly. Embodiments may include blood specimen collection tubes or vacutainers and tube holders. The parts of syringes and other blood collection items may be injection molded using the plant based furan resins. Embodiments of furan based syringes may be used for insulin administration, allergy or tuberculin testing or administration of other parental agents.

An embodiment of a syringe may have a plunger that fits tightly in a tubular barrel with a tip. The plunger may be pulled and pushed along inside a cylindrical tube, the barrel, allowing the syringe to take in and expel a liquid or gas through an orifice at the open end of the tube called the tip. The open end of the syringe or the tip may be fitted with a hypodermic needle. The tip may be of various shapes. A cone shape or threaded embodiment may allow for a needle or tubing to be attached, by simple pushing or screwing, the latter by circular twisting motion. This attachment may help direct the flow into and out of the barrel. The needle head is called a hub and an opening or aperture. Embodiments of furan based syringes may be used in the medical field to administer injections, insulin administration, skin tests such as allergy tests, and tuberculin testing. In non-medical field uses, non-sterile furan based syringes may be used to apply compounds such as glue or lubricant, and measure liquids.

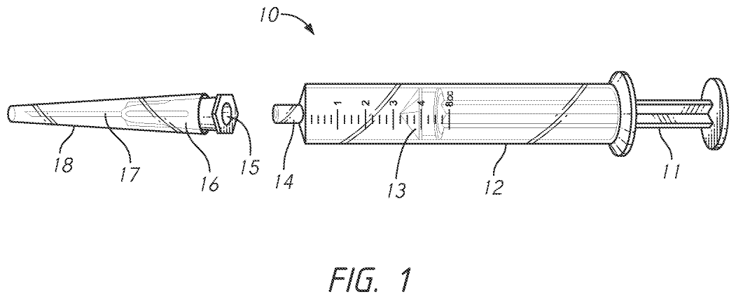

FIG. 1 depicts a perspective view of a general propose disposable single-use syringe 10 that may has a plunger 11 with a rubber tip 13, a cylindrical barrel 12, a hypodermic needle 17 and a needle cap 18. The barrel 12 has a tip 14. The needle 17 may have a hub 16 and opening or aperture 15.

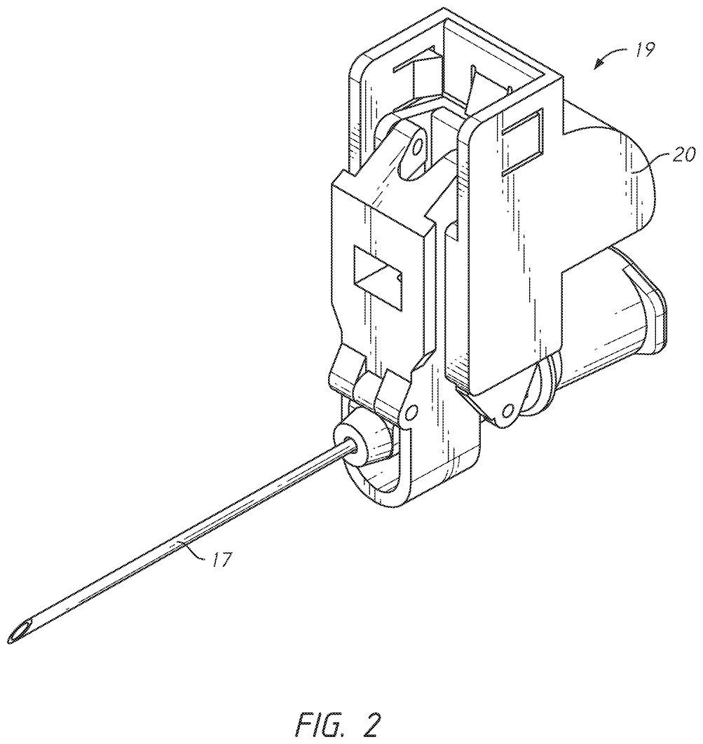

FIG. 2 depicts needle and safety shield assembly 19, which may include a needle 17 and a shield assembly 20.

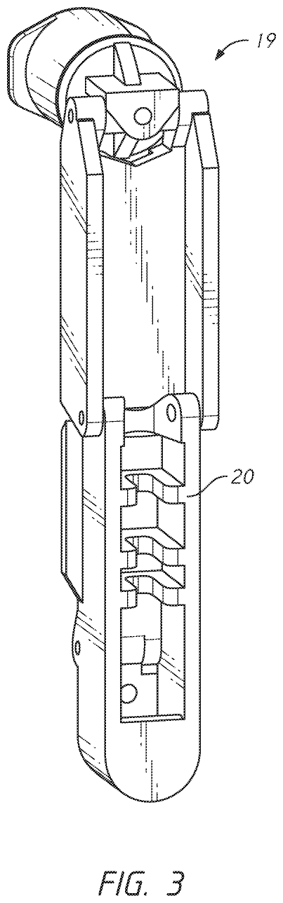

FIG. 3 depicts an embodiment of a safety needle and shield assembly 19 without the needle in an extended position.

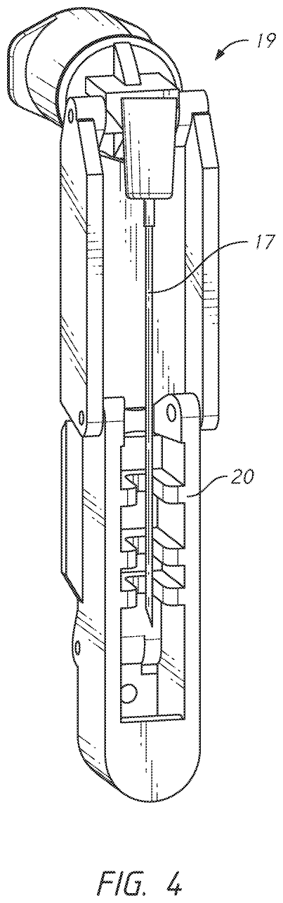

FIG. 4 depicts an embodiment of a safety needle and shield assembly 19 with the needle in an extended position, which may accommodate the needle in a protective casing.

An embodiment of a syringe safety needle assembly 19 may include a syringe 10, fixed to a medical or hypodermic needle 17. An embodiment of a furan resin cap and a safety shield assembly 20 may attach to the syringe needle assembly 19, to help provide protection from a sharpened tip of the needle 17. The cap 18 may be removed before using the syringe needle assembly. After using the syringe needle assembly 19, the safety shield 20 may be deployed. The safety shield 19 may be foldable or may include a tubular assembly 18, thereby providing a safety sheath for the needle. This may help guard against problems associated with inadvertent needle sticks related to blood sampling, percutaneous medication injection and other medical procedures involving uses of medical needles.

Embodiments of the syringe and related parts 11, 12, 14, 16, and 18 may be injection molded using the furan resins.

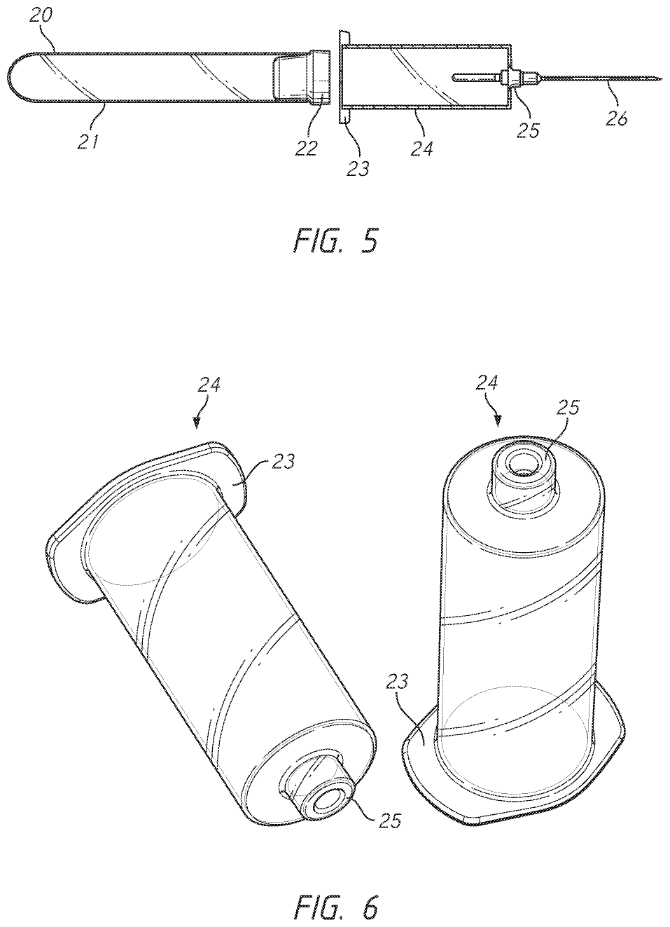

FIG. 5 depicts an embodiment of blood collection items collection tube 20, with vial 21 and rubber stopper 22, a tube holder 24 that has flange 23, and needle tip 25. A needle 26 is fitted o the needle tip 25.

FIG. 6 depicts an embodiment of a tube holder 24 with open distal end with flange 23 and the tapered proximal with needle tip 25. Embodiments of tube holder 24 may be injection molded using furan resins.

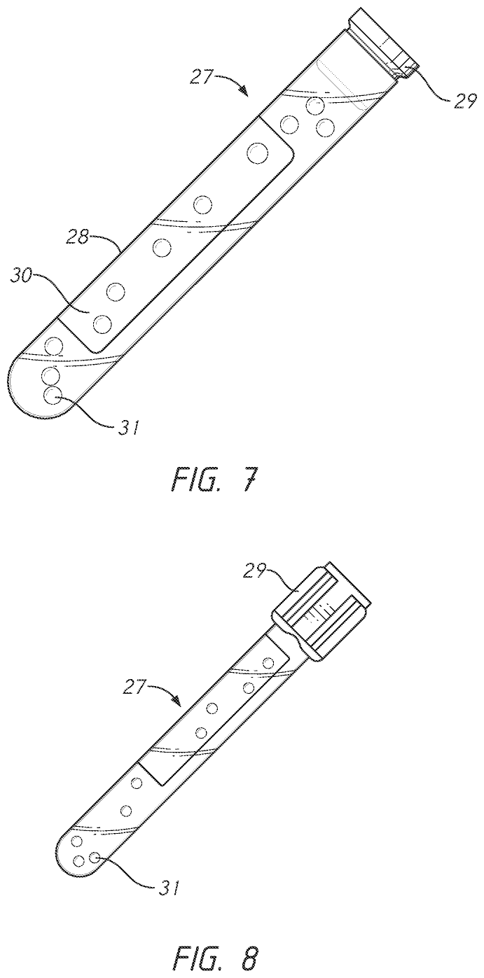

FIG. 7 depicts an embodiment of a blood collection tube or vacutainer 27, with vial 28, rubber stopper 29, and label 30. A space 31 may provide a vacuum to allow blood or fluid to flow during a venipuncture. Embodiments of vial 28 may be injection molded using furan resins.

FIG. 8 depicts an embodiment of a blood collection tube or vacutainer 27 with a cap 29 that acts like a rubber stopper.

Embodiments of blood collection items such as vial 28 and tube holder 24 may be injection molded using furan resins.

I claim:

1. A blood collection item comprising a furan resin selected from the group consisting of:
poly (ethylene 2,5-furan dicarboxylate) (PEF),
poly (butylene 2,5-furan dicarboxylate) (PBF),
poly (trim ethylene furan dicarboxylate) (PTF),
poly (propylene 2,5-furandicarboxylate) (PPF), and
poly (neopentyl 2,5-furandicarboxylate) (PNF).

2. The blood collection item of claim 1 wherein the blood collection item is injection-molded using the furan resin.

3. The blood collection item of claim 1 wherein the blood collection item is a syringe.

4. The blood collection item of claim 1 wherein the blood collection item is a component for a syringe and the component is injection-molded using the furan resin.

5. The blood collection item of claim 1 wherein the blood collection item is a plunger for a syringe.

6. The blood collection item of claim 1 wherein the blood collection item is a needle cap for a syringe.

7. The blood collection item of claim 1 wherein the blood collection item is a barrel for a syringe.

8. The blood collection item of claim 1 wherein the blood collection item is a barrel tip for a syringe.

9. The blood collection item of claim 1 wherein the blood collection item is a blood collection tube.

10. The blood collection item of claim 1 wherein the blood collection item is a vial for a blood collection tube.

11. The blood collection item of claim 1 wherein the blood collection item is a tube holder for a blood collection tube.

12. The blood collection item of claim 1 wherein the blood collection item is a blood collection tube formed by injection molding using the furan resin.

13. The blood collection item of claim 3, wherein the furan resin is PEF.

14. The blood collection item of claim 3, wherein the furan resin is PBF.

15. The blood collection item of claim 3, wherein the furan resin is PTF.

16. The blood collection item of claim 3, wherein the furan resin is PPF.

17. The blood collection item of claim 3, wherein the furan resin is PNF.

18. A syringe comprising:
a furan-based resin that includes at least one of:
poly (ethylene 2,5-furan dicarboxylate) (PEF),
poly (butylene 2,5-furan dicarboxylate) (PBF),
poly (trim ethylene furan dicarboxylate) (PTF),
poly (propylene 2,5-furandicarboxylate) (PPF), or
poly (neopentyl 2,5-furandicarboxylate) (PNF);
an injection-molded barrel that includes the furan-based resin; and
an injection-molded plunger that includes the furan-based resin.
